(19) United States
(12) Patent Application Publication
(10) Pub. No.: US 2009/0264941 A1
(43) Pub. Date: Oct. 22, 2009

(54) GUIDE SLEEVE FOR ACCESSING A VERTEBRAL BODY AND RELATED METHODS OF USE

(76) Inventor: EZZINE BANOUSKOU, TOULOWSE (FR)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/417,555

(22) Filed: Apr. 2, 2009

Related U.S. Application Data
(63) Continuation of application No. PCT/IB2008/002294, filed on Apr. 8, 2008.

(51) Int. Cl. A61F 5/00; A61F 11/00

(52) U.S. Cl. 606/86 R; 606/108

(57) ABSTRACT

Embodiments of the subject disclosure are directed toward surgical devices, and more particularly, to a guide sleeve apparatus and its use thereof, where the guide sleeve apparatus provides access to a vertebral body for use in various therapeutic treatments. Methods of using such guide sleeves for delivering devices and agents via the access provided by the sleeve, as well as methods of employing the guide sleeve in performing various therapeutic treatments, are also provided.

### RELATED APPLICATIONS

This application is a continuation of PCT Application No. PCT/IB2008/002294 filed Apr. 8, 2008 under 35 USC §111(a). Priority of the aforementioned filing date is hereby claimed, and the disclosures of the PCT Patent Application is hereby incorporated by reference in its entirety.

### FIELD OF THE DISCLOSURE

The present disclosure relates to surgical devices, and more particularly, to a guide sleeve apparatus and its use thereof, where the guide sleeve apparatus provides access to the vertebra, e.g., the entire vertebra or a portion thereof, for use in various therapeutic treatments.

### BACKGROUND OF THE DISCLOSURE

The spinal column is generally made up of three main components: the spinal cord, vertebrae, and intervertebral discs. The vertebrae are stacked on top of each other, and are provided from the bottom of the skull to the pelvis. Each vertebra is generally composed of several parts that act as a whole to surround and protect the spinal cord and nerves, provide structure to the body, and enable fluid movement of the body in many planes.

A compression fracture occurs when a normal vertebral body of the spine is compressed to a smaller height. This injury tends to happen in two groups of people: those who are involved in a traumatic accident, such as a fall, and those who are suffering from osteoporosis. Osteoporosis is a condition that causes a thinning of the bone. Specifically, as the bone thins it is less able to support a load, and thus, when a load placed on the vertebrae exceeds its stability, it collapses. In addition, certain diseases, such as cancer, are known to cause loss of bone mass and changes in bone structure, which can effect the vertebrae by making them brittle and weak. Genetic factors and certain lifestyles, such as a low calcium diet, can also result in damage to the vertebral bones. Over time, the vertebral bodies can become so weak that normal activities such as bending over or lifting a bag of groceries, can cause a spinal fracture and collapse of the vertebral body.

Collapse of a vertebral body occurs when there is a sudden increase in loading beyond which the vertebral bone can tolerate. Such collapse causes severe pain because the membrane surrounding the vertebral bone, the periosteum, is richly innervated with nerve fibers that can become pinched by the collapse of the vertebral body and thereby generate pain signals. Further, the vertebral body collapse can cause a loss of contained vertebral bone marrow, resulting in further deterioration of the spinal column, and an associated loss of vertebral body height, leading to further spinal compression.

If left untreated vertebral fractures can lead to spinal deformities, such as: a curvature of the spine (e.g., scoliosis and/or dowager's hump syndrome), spinal compression and/or a loss of length of the spinal column, deterioration of the vertebrae, and eventually the entire or partial collapse of the vertebral body and/or spinal column. Specifically, in the instance of vertebral deterioration caused by osteoporosis, once the osteoporotic bones of a vertebral body have become soft enough to develop vertebral fractures under a relatively normal load, other vertebral bodies may soon develop stress fractures the collapse of which may result in the overall collapse of the spinal column.

Typically, the treatment of vertebral compression fractures has been limited to the administration of pain medicine, bracing, resting, and otherwise taking care to avoid spinal injury. Surgical techniques, e.g., vertebroplasty and kyphoplasty, have been developed to remove and/or reduce pressure on the spinal nerves caused by the mechanical breakdown of the spinal column due to the collapse of the vertebral body. Several vertebroplasty techniques are now practiced for effectuating vertebral correction e.g., to substantially restore a vertebral body to its original shape. Generally, vertebroplasty is a surgical procedure typically used for a spinal fracture, e.g., such as those caused by osteoporosis, where a glue-like material or bone cement (e.g., methylmethacrylate) is percutaneously injected into a fractured vertebral body in order to stabilize it.

Kyphoplasty is another surgical technique. In one type of kyphoplasty procedure, an inflatable balloon is implanted into a vertebra, then fluid under pressure is introduced into the balloon, which in turn compacts cancerous bone outward and also forces cortical bone of the vertebra, and in particular the lower and upper vertebral plateaus, to correct the shape of the vertebra under the effect of the pressure. Once the osseous cortical shell has been corrected, the balloon is deflated, withdrawn from the vertebra, and a cement is injected into the vertebral body so as to impart sufficient mechanical resistance for the correction.

The effectiveness of vertebroplasty and kyphoplasty depends in part on the design and usability of the instrumentation employed in performing the procedure so as to correct vertebral fractures within the vertebral body. During such procedures, it is essential that the instrumentation be properly and stably positioned within the vertebral body at the precise point in need of treatment. As such, it is desirable to continue to improve devices and the methods of their use for accessing the vertebral body so as to more effectively position devices, such as such vertebroplasty and kyphoplasty devices, for their use in performing various therapeutic treatments. Embodiments of the subject disclosure presented herein meet these and other needs in the art.

### SUMMARY OF THE DISCLOSURE

Accordingly, embodiments of the present disclosure provide systems, apparatuses, devices and methods for accessing and/or guiding a medical instrument to a bone. In particular, some of the embodiments of the subject disclosure are directed to a guide sleeve, which may be inserted into a portion of a vertebral body (for example) so as to provide percutaneous access thereto. In some embodiments, the guide sleeve may include an elongate tubular body having a distal portion with a distal end, a proximal portion with a proximal end, and a lumen between the distal and proximal ends. The guide sleeve may further include a penetration member. The penetration member may be positioned on the distal portion of the sleeve and may be configured for penetrating a bone portion, for instance, a vertebral bone portion. The guide sleeve may additionally include a fixation element. The fixation element may be positioned at the distal portion of the guide sleeve and may be configured for fixing the sleeve to a bone portion. In certain embodiments, the guide sleeve may be inserted and affixed to the bone portion by rotating the sleeve. Methods of using such guide sleeves, methods of treatment, and methods for delivering devices and agents via the access provided by the sleeve, as well as methods of employing the guide sleeve in performing various therapeutic treatments, are also provided.

### BRIEF DESCRIPTIONS OF THE DRAWINGS

The figures shown herein are not necessarily drawn to scale, with some components and features being exaggerated for clarity.

FIG. 1 provides an exemplary embodiment of the subject guide sleeves.

FIG. 2 provides a detail view of an exemplary embodiment of the guide sleeve penetration member, wherein the penetration member is configured as a tooth or teeth.

FIG. 3, panels A-E, illustrate the guide sleeve penetration member configured to include a penetration tooth or teeth positioned symmetrically at a distal end of the subject guide sleeves.

FIG. 4 provides a detail view of an exemplary embodiment of a fixation element configured as a threaded portion positioned at a distal portion of a guide sleeve.

FIG. 5 provides a detail view of an exemplary embodiment of the proximal portion of the subject guide sleeves.

FIG. 6, panels A-D, illustrate the use of the subject guide sleeve in conjunction with various other instruments.

FIG. 7 provides an exemplary embodiment of a handle attached to a proximal portion of the subject guide sleeves.

FIG. 8, panels A-D, provides an exemplary embodiment of the application of the subject guide sleeves.

FIG. 9, panels A-D, provides additional exemplary embodiments of the application of the subject guide sleeves.

### DETAILED DESCRIPTION OF THE DISCLOSURE

Before the present disclosure is described, it is to be understood that the disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value between the upper and lower limit of that range (which may be down to the tenth of the unit of the lower limit unless the context clearly dictates otherwise), and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

Unless defined otherwise, all technical and scientific terms used herein have the same meaning as commonly understood by one of ordinary skill in the art to which the present disclosure belongs. Although any methods and materials similar or equivalent to those described herein can also be used in the practice or testing of embodiments of the present disclosure, some of the preferred methods and materials are now described.

It must be noted that as used herein and in the appended claims, the singular forms “a”, “an”, and “the” include plural referents unless the context clearly dictates otherwise. Thus, for example, reference to “a penetration member” includes a plurality of such members, and reference to “the fixation element” includes reference to one or more fixation elements and equivalents thereof known to those skilled in the art, and so forth.

In the following discussion, the terms “proximal” and “distal” will be used to describe the opposing axial ends of the inventive apparatus, as well as the axial ends of various component features of the apparatus. The term “proximal” is used in its conventional sense to refer to the end of the apparatus (or component) that is handled by, or is closest to the operator during use of the guide sleeve. The term “distal” is used in its conventional sense to refer to the end of the apparatus (or component) that is initially inserted into the patient, or that is closest to the patient.

All publications mentioned herein are incorporated herein by reference which may disclose and describe methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the embodiments described and/or illustrated herein has discrete components and features which may be readily separated from or combined with the features of any of the other several embodiments without departing from the scope or spirit of the present disclosure. Any recited method can be carried out in the order of events recited or in any other order which is logically possible.

Some of the embodiments of the present disclosure provide a guide sleeve which may be inserted into a portion of a vertebra so as to provide percutaneous access thereto. In some embodiments, the guide sleeve includes an elongate tubular body having a distal portion with a distal end, a proximal portion with a proximal end, and a lumen between the distal and proximal ends. The guide sleeve may further include a penetration member. The penetration member may be positioned on the distal portion of the sleeve and may be configured for penetrating a bone portion, for instance, a vertebral bone portion. The guide sleeve may additionally include a fixation element. The fixation element may be positioned at the distal portion of the guide sleeve and may be configured for fixing the sleeve to a bone portion. In certain embodiments, the guide sleeve may be inserted and affixed to the bone portion by rotating the sleeve. Methods of using such guide sleeves, as well as methods for delivering devices and agents via the access provided by the sleeve, as well as methods of employing the guide sleeve in performing various therapeutic treatments, are also provided herein.

Some embodiments of the subject disclosure enable the delivery or positioning of instrumentation within the vertebral body at a point in need of treatment using a guide sleeve. As such, the guide sleeve preferably provides percutaneous access thereto when inserted into a portion of a vertebral body. In addition, in some embodiments, the guide sleeve is not only configured to receive other instruments and agents, but also serves to guide such instruments and agents through the guide sleeve in proper alignment to a specific target site within a vertebral body.

The subject guide sleeve may be formed from conventional materials and by methods well known and used in the art. The guide sleeve need not be rigid though preferably includes sufficient rigidity such that it does not yield to substantial bending/deforming, or yields only in a minor amount, when utilized. Non-limiting examples of suitable materials for manufacturing the subject guide sleeves include conventional metals and metal alloys, such as stainless steel, titanium, nickel-titanium, steel alloys or plastic/composite members, such as PTFE, polycarbonates, nylon, polypropylene, PEEK, and acetal. The subject guide sleeve may be manufactured by any methods well known in the arts, such as by turning, milling, molding, cutting, electrical discharge machining (EDM), and the like.

In some embodiments, the guide sleeve may include an elongate tubular body having a distal portion with a distal end and a proximal portion with a proximal end. The elongate tubular body includes an inner lumen positioned between the distal and proximal ends. The elongate tubular body of the subject guide sleeve may have an external diameter ranging from about 3.0 mm to about 30.0 mm (or greater), and in some embodiments, from about 4.0 mm to about 8.0 mm; and, in some embodiments, including from about 5.0 mm to about 7.5 mm. Further, the length of the subject guide sleeve's elongate tubular body may range from about 60.0 mm to about 250.0 mm (or greater), and in some embodiments, from about 100.0 mm to about 200.0 mm; and in some embodiments, from about 130.0 mm to about 170.0 mm.

The elongate tubular body of the subject guide sleeve may further include an internal lumen which is configured in such a manner that an additional device or agent may be guided there through. The internal lumen of the subject guide sleeve may have a diameter ranging from about 2.0 mm to about 9.0 mm, and in some embodiments, from about 3.5 mm to about 7.0 mm; and in some embodiments, from about 5.0 mm to about 6.0 mm. In some embodiments, the internal lumen is configured for receiving other devices and instruments, for example, a vertebroplasty device, a kyphoplasty device, intersomatic cages, screws, spacers and the like. The internal lumen may have an elongated, cylindrical dimension wherein the surface that bounds the lumen may be smooth. In some embodiments, the surface that bounds the lumen may include a configuration that is adapted to engage and/or retain a device or agent inserted there through within a longitudinal axis of the lumen. In some embodiments, the internal surface that bounds the lumen may be threaded or include other configurations and/or elements that serve to enhance or control the guiding of an instrument through the lumen of the guide sleeve, such as a groove, slot, thread, flange, a combination thereof and the like.

In some embodiments, the internal lumen may include a visualization component. For example, a visualization component may be included to allow the treatment or procedure to be visualized, such as, via fluoroscopy. For instance, radio transparent materials, such as plastics, may be included in connection with the subject guide sleeve so as to promote visualization of the guide sleeve during its use in a treatment or procedure. Visualization of the lumen may also be enhanced by inclusion of a radiopaque tracking device on the distal end of the tubular elongate body which allows the depth of penetration of the guide sleeve to be monitored by one or more depth markings.

In some embodiments, the subject guide sleeve may include a penetration member positioned at the distal portion of the sleeve, which may be configured for penetrating a bone or tissue portion of the patient, for instance, a vertebral bone portion.

In some embodiments, the guide sleeve includes a fixation element, which may be positioned at the distal portion of the subject guide sleeve. The fixation element may be configured for fixing the sleeve to a portion of the patient's body, such as a bone portion. For instance, in some embodiments, the fixation element is configured such that by rotating the subject guide sleeve, the distal portion of the sleeve containing the fixation element engages and becomes affixed/attached to a portion of the patient's body, for instance, at a specific target site within a vertebral body.

In some embodiments, a subject guide sleeve may include both a penetration member and a fixation element positioned at a distal portion of the guide sleeve. The penetration member and fixation element may operatively be aligned such that the subject guide sleeve may be rotated in a manner so that the penetration member penetrates a portion of the patient's body and the fixation element attaches and fixes the guide sleeve to said portion, for instance, a specific bone portion in need of treatment.

In some embodiments, the guide sleeve may include an attachment member positioned at a proximal portion of the elongated tubular body of the sleeve. The attachment member may be adapted for receiving an instrument member and thereby associating the instrument member with the guide sleeve. An attachment member may have any suitable configuration so long as it is capable of engaging a portion of an instrument member and thereby affixing a portion of the instrument member to the guide sleeve for use therewith. Hence, in some embodiments, an attachment member is a proximal portion configuration or element that is adapted for allowing and/or facilitating the attachment of an instrument member to the guide sleeve. The proximal portion may be configured to receive other devices and instruments, for example, a vertebroplasty device, kyphoplasty device, intersomatic cages, screws, spacers and the like. In some embodiments, the proximal portion of the subject guide sleeve may be configured so as to attach to an additional device or instrument, for example, an awl, drill, template, implant holder, cement injection tube, or the like to the guide sleeve, e.g., at a proximal end of the guide sleeve.

The penetration member may have any suitable configuration so long as it is capable of penetrating one or more of a tissue, cartilage, and/or bone portion of a subject's body and thereby allowing a portion of the guide sleeve to penetrate and/or engage said body portion. For example, the penetration member may be a configuration on the distal portion, e.g., distal end, of the guide sleeve and be adapted so as to penetrate tissue and/or bone. In some embodiments, the penetration member may be a configuration that includes one or more of a sharp edge configuration, a serrated edge configuration, a configuration containing one or more sharp pointed elements, a configuration including a tooth or teeth, a combination thereof, and the like. The penetration member may also be coextensive with the elongate tubular body.

In some embodiments, the penetration member may be a separate component that is attached to the elongate tubular body, such as a sharpened blade member, a pointed puncture member, and the like. Hence, in some embodiments, a penetration member is a configuration positioned at the distal portion of the guide sleeve and adapted for penetrating a body portion of a subject, for instance, a vertebral body of a subject's spinal column.

In some embodiments, the penetration member may be configured as one or more teeth. Specifically, the penetration member may include one or more cut-outs, for instance, a U-shaped cut-out, which U-shaped cut out is in the configuration of one or more teeth, for example, with the one or more teeth being formed at the distal end of the subject guide sleeve. The cut-out may include a sharp edge portion, for instance, a portion that is sharp enough to penetrate into and/or affix the subject guide sleeve to a specific target site within a vertebral body.

A suitable tooth (or teeth) of the penetration member may have a height ranging from about 0.2 mm to 20 mm, and in some embodiments from about 3 mm to about 15 mm; and in other embodiments, from about 5 mm to about 10 mm. In addition, the teeth may have a width ranging from about 0.2 mm to 5 mm, and in some embodiments, from about 1 mm to 4 mm; and in some embodiments, from about 2 mm to 3 mm. Further, the distances between the teeth may range from about 0 mm to about 5 mm, and in further embodiments, from about 0.5 mm to about 3 mm, including from about 1 mm to about 2 mm. A suitable penetration member of the present disclosure may be manufactured using a variety of processes known in the art. For example, the penetration member may be manufactured by milling, lathing, casting, EDM, or the like.

In some embodiments, the guide sleeve includes a fixation element, which may be positioned at a distal portion of the elongated tubular body of the guide sleeve. A fixation element may have any suitable configuration that may enable it of engaging one or more of tissue, cartilage, and/or bone portion of a subject's body and thereby affixing a portion of the guide sleeve to said body portion. Hence, in some embodiments, a fixation element is configured for being affixed to a bone portion of a subject, for instance, a vertebral body of a subject's spinal column.

In some embodiments, a suitable fixation element may be configured as one or more screw threadings. In some embodiments, the fixation element may include a single thread or multiple threads. A suitable threaded portion may have various configurations, for example, a bone thread configuration, a buttress thread configuration, a fish-hook thread configuration, anchor-type shape thread configuration, a square thread configuration, a mixture of the above, and the like. Moreover, the thread may have any suitable pitch, for instance the threading may have a pitch that ranges from about 0.5 mm to about 10 mm, including about 1 mm to about 8 mm, such as from about 2 mm or 4 mm to about 6 mm. For instance, in some embodiments, the threading has a pitch of 5 mm. In general, the shape and the size of threads may be chosen based on factors such as the cohesiveness of the material from which threads are formed, and the coefficient of friction associated with the material. The shape and the size of threads are also typically chosen based upon the type of bone material through which the threads are intended to penetrate. For example, in some embodiments, the one or more threads may have a shape selected from the group consisting of: 60°, square, buttress, bone thread specific, symmetric, non-symmetric, and the like. In some embodiments, the threading may have a height that ranges from about 0.2 mm to about 3 mm, such as from about 0.5 mm to about 2 mm, including about 1 mm to about 1.5 mm.

In some embodiments, the threading may be symmetrical or asymmetrical, where asymmetric threads enable the guide sleeve to be effectively controlled. In addition, asymmetric threads, such as buttress-type threads, may enable the distal portion of the guide sleeve to be relatively easily driven through bone, while preventing the guide sleeve from being pulled or backed out of the bone. Additionally, the presence of asymmetric threads facilitates the positioning of the distal portion by effectively allowing a certain amount of rotational motion to translate into a certain amount of linear motion. The presence of threads also enables controlled positioning of the guide sleeve through rotational motion.

The attachment member may be positioned at a proximal portion of the elongated tubular body of the guide sleeve. For instance, in some embodiments, the guide sleeve includes an attachment member configuration that is adapted for receiving at least a portion of a surgical instrument member and thereby associating the instrument member with the guide sleeve. Such an attachment member may have any suitable configuration for enabling it to engage a portion of an instrument and thereby affixing a portion of the instrument to the guide sleeve for use therewith. Hence, in some embodiments, the attachment member is a proximal portion configuration of the guide sleeve that is adapted for allowing and/or facilitating the attachment of an instrument (surgical or otherwise) to the guide sleeve.

For example, the attachment member may comprise an opening that is configured to be mated with an insertion portion of instrument. Specifically, the attachment member may be configured as a female receptacle portion that is adapted to receive a male insertion portion of an instrument. In other embodiments, the attachment member may be configured as a male insertion member that is adapted to be received within a female receptacle portion of an instrument to be connected therewith. A suitable attachment member may have any suitable shape or any suitable size, including round, circular, triangular, square, pentagonal, hexagonal, or other such shape. The attachment member may be tubular and concave comprising a lumen with a given shape or it may be tubular and extended.

In some embodiments, a suitable attachment member may include a lumen, where the lumen is bounded by portions of the proximal portion of the guide sleeve, for instance, in the shape of a square or hexagon. The square or hexagonal shape may have a size that ranges from 2.5 mm to about 9 mm, for instance, about 4 mm to about 8 mm, including about 5 mm to about 6 mm. The proximal portion bounding the lumen may additionally include threading. One or more threads may be included. In some embodiments, the one or more threads may have a shape selected from the group consisting of: 60°, square, buttress, bone thread specific, symmetric, non-symmetric, and the like. In some embodiments, the thread may have a height that ranges from about 0.2 mm to about 3 mm, such as from about 0.5 mm to about 2 mm, including about 1 mm to about 1.5 mm. Further, the thread may have any suitable pitch, for instance, the threading may have a pitch that ranges from about 0.5 mm to about 5 mm, including about 1 mm to about 4 mm, such as from about 2 mm to about 3 mm. For instance, in certain embodiments, the threading has a pitch of 2.5 mm.

In some embodiments, the attachment element includes a lumen where the lumen is bounded by one or more surfaces that include an internal configuration adapted for receiving and attaching an insertion portion of an apparatus to the guide sleeve. For example, the interior surface may include screw threading, which threading corresponds to threading on an insertion portion of an instrument whereby the instrument may be associated to the guide sleeve by the threading of the insertion member and the attachment member together.

In this or any similar manner any of a number of instruments may be associated with the guide sleeve. For instance, a suitable instrument that may be removably coupled to the guide sleeve includes a handle. For example, a handle containing s suitable insertion portion, e.g., a threaded insertion portion, may be attached to the guide sleeve by threading the insertion portion into the receiving portion of the attachment member.

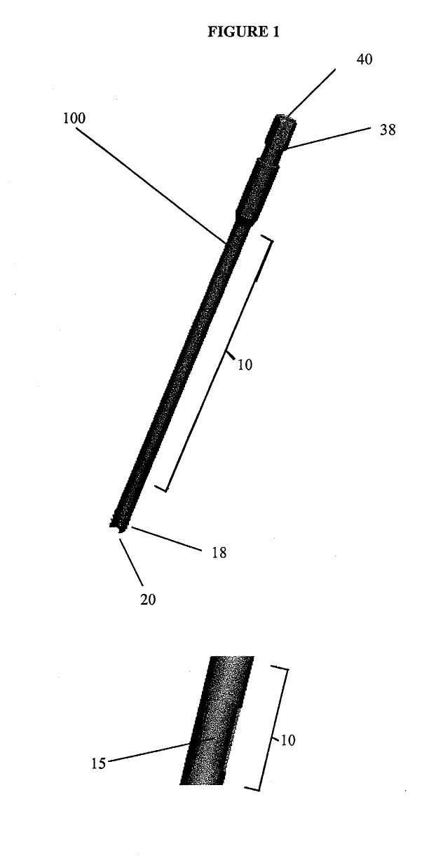

As summarized above, the guide sleeves of the present disclosure may be employed so as to provide percutaneous access to a vertebral body, iliac crest, or other bone for delivering devices and agents thereto via the access. Reference will now be made in detail to various embodiments of the disclosure, which are illustrated in the accompanying figures. Referring now to FIGS. 1-9, the guide sleeve (100) includes an elongate body (10) which includes a distal portion (18) and a distal end (20), as well as a proximal portion (38) and a proximal end (40). The elongate body (10) is tubular and includes an inner lumen (15). The inner lumen (15) is configured to receive one or more instruments and/or agents and is also configured to guide such instrument(s) and agent(s) through the guide sleeve in proper alignment. In some embodiments, the elongate body may include a cleft.

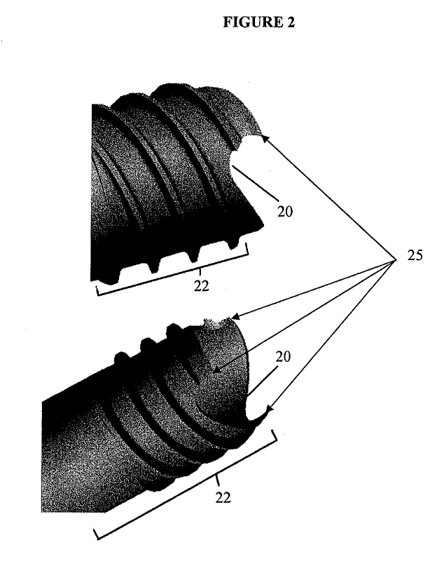

With reference to FIG. 2, in certain embodiments, the distal portion (18), e.g., the distal end (20), of the guide sleeve may include a penetration member (25) which enables the distal end (20) of the guide sleeve to penetrate into a body portion such as bone, e.g., the bone of a vertebral body, the bone of the iliac crest, or any other bone. In some embodiments, the penetration member (25) is coextensive with the elongate tubular body (10) and may include one or more anchoring members or teeth as depicted in FIG. 2. As illustrated, the one or more teeth may be configured as a cut out portion of the distal end of the guide sleeve's elongated tubular body (10). The one or more teeth may be a separate component from the subject guide sleeve, and may be made of a different material from the main tubular member of the subject guide sleeves and be configured for attachment via suitable coupling configuration to the distal end of the guide sleeve.

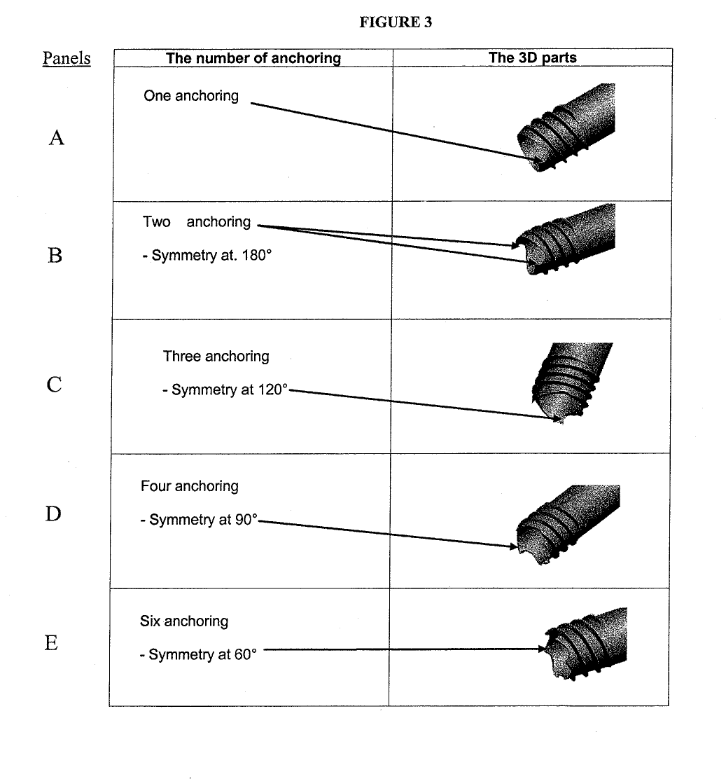

In embodiments having one, two, or more teeth, the teeth may or may not be symmetrically positioned at the distal end (20) of the subject guide sleeves. For instance, as illustrated in FIG. 3, panels A-E the distal end (20) of the sleeve may be symmetrical. For example, when the subject guide sleeve has two teeth, the teeth at the distal end of the subject guide sleeves may be symmetrically positioned at 180° angles. When the subject guide sleeves have three teeth at the distal end, the teeth may be symmetrically positioned at 120°. Where the subject guide sleeves have four teeth at the distal end, the teeth may be symmetrically positioned at 90° angles; and the like. Where the guide sleeves of the present disclosure are rotated, the penetration member penetrates into the body portion, e.g., bone, until the subject guide sleeves are inserted therein.

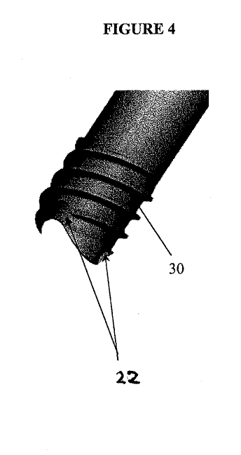

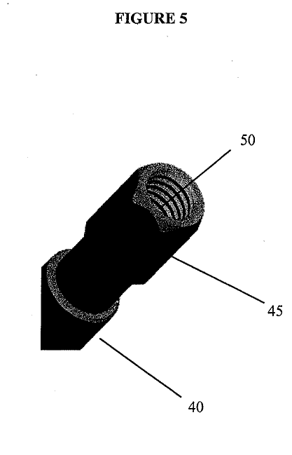

The distal end (20) of the guide sleeve (10) may further include a fixation element (22) which enables the guide sleeve to attach/affix to the bone. The fixation element (22) may include a threaded portion (30) as illustrated in FIG. 4, which may be a single thread or multiple threads, which are coextensive with the elongate tubular body and the penetration member (25). Accordingly, when the guide sleeves of the present disclosure are rotated, the fixation element (22) engages into the bone as a result of the helicoidal shape of the thread until the subject guide sleeves are locked, thereby resulting in the attachment/affixation of the guide sleeve into the bone.

As such, when the guide sleeves of the present disclosure are rotated, the penetration member penetrates into the body portion, e.g., bone, until the subject guide sleeves are inserted therein. Additionally, as the guide sleeve may be further rotated, the fixation element, positioned at the distal portion of the guide sleeve, may be rotated, thereby engaging the body portion such that the fixation element attaches to the body portion, e.g., the specific bone portion in need of treatment. For example, the rotational motion causes the threaded portion positioned at the distal portion of the subject guide sleeve to rotate into the body portion, e.g., bone, and become affixed/attached thereto, for instance, at a specific treatment site.

Accordingly, the above described penetration members and fixation elements enable the subject guide sleeves to be inserted and/or affixed directly into the bone by rotational motion. As the guide sleeve is rotated, for example by a handle coupled to the proximal portion of the guide sleeve, the distal portion of the guide sleeve penetrates into the bone in a distal direction. Therefore, the subject guide sleeves may be precisely and stably positioned within a bone structure. Further, the penetration members and fixation elements not only serve for purposes of attachment and fixation, but further add stability to the guide sleeve by preventing the guide sleeve from retracting in a proximal direction.

As illustrated in FIG. 5, the guide sleeves according to some embodiments may additionally include an attachment member (45) at the proximal portion (38), e.g., the proximal end (40), wherein the attachment member (45) is configured for allowing and/or facilitating the association of the tubular body (10) with an extrinsic instrument member. For instance, in certain embodiments, the guide sleeve (100) may include an attachment member (45) that is configured to facilitate the specific attachment of a handle, an additional instrument, or a connector element to the proximal portion (38) of the guide sleeve (100).

To that end, the proximal end may be configured to have a specific external shape. For example, in the embodiment illustrated in FIG. 5, the proximal end (40) has a hexagonal shape. In some embodiments, the hexagonal shape may comprise a hex-nut configuration. Any handle or additional instrument or connector element may therefore have a complimentary hexagonal shape in order for attachment to occur and to avoid relative rotation and/or relative torsion. In other embodiments, the proximal end may be configured to have a plane, a groove or multiple grooves, a triangular shape, a square shape, or any polygonal shape and/or additionally include a thread-like configuration. These shape configurations can be combined together to provide a connection between the guide sleeve and the device to be attached to it.

The proximal end (40) may have an internal configuration for receiving additional devices or agents that have a complementary shape thereto. As depicted in FIG. 5, the proximal end (40) has a circular (50) internal shape. Thus, an additional device or agent to be associated with the proximal end (40) may have a complementary shape to the internal shape of the proximal end (e.g., in this instance, circular), in order for the additional device or agent to be associated with the proximal end attachment member. For example, where the attachment member and additional instrument to be associated therewith have complimentary male to female configurations, the end portions of the two devices may be inserted one into the other. For example, where a portion of an additional instrument includes a male attachment element, this element may be received within the attachment member (45) of the tubular body (10) and inserted into the inner lumen (15) via the proximal end (40) of the guide sleeve.

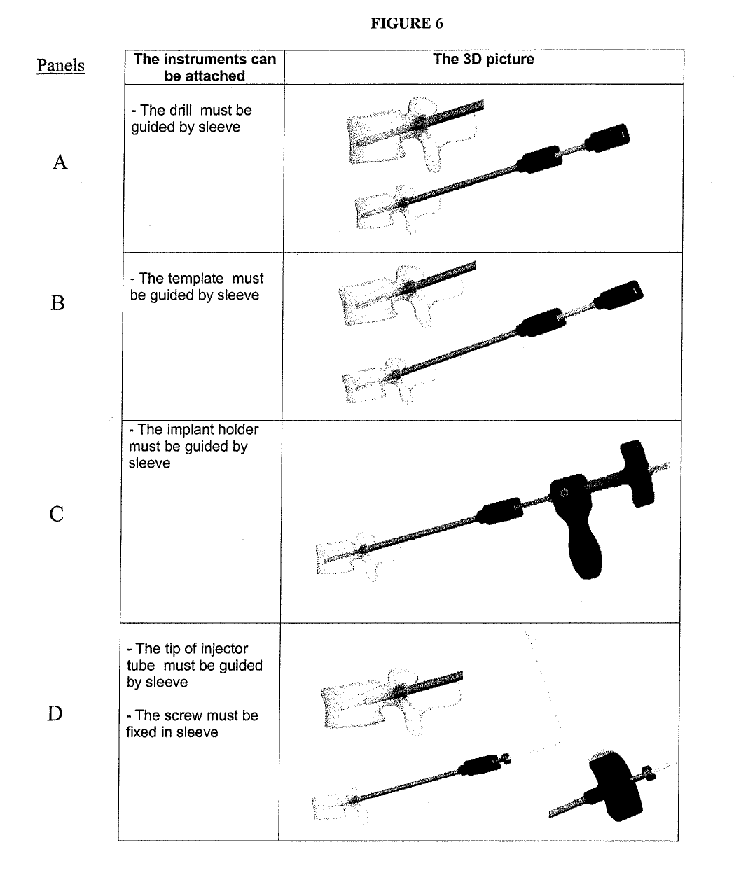

Exemplary devices or instruments which may be linked to the proximal end of the subject guide sleeves include, but are not limited to the following: an awl, for forming a hole for the insertion of a screw or other device in a pedicle or other body part; a drill, for creating a hole in a vertebra with high accuracy to place pedicle screws; a reamer, for creating a smooth surface to introduce an intravertebral implant or instrument to be adjusted to the hole performed; a template, for determining the width of the space between two bodies, such as the vertebral bodies; an implant holder, for securing a vertebral implant; a cement injection tube, for vertebroplasty or kyphoplasty procedures, and the like. FIG. 6, panels A-D illustrate the attachment of such instruments to the subject guide sleeves. For instance, an awl, a drill, a reamer, or a template, etc. may be guided through a cylindrical shape allowing a translation and rotation motion between the proximal end and the awl, etc. Further, a cement injection tube may be attached to the proximal end by screwing or using a clamp.

In some embodiments, the proximal portion of the guide sleeve may further include an attachment member (45) that is configured to have a specific external shape. For instance, the attachment member may comprise a male insertion element that is adapted for being received within a female receptacle element of an instrument member. Specifically, the external attachment member may include an exterior configuration that has one or more walls of the proximal portion of the guide sleeve, where the wall(s) are configured as a particular shape, such as a circle, triangle, square, rectangle, pentagon, hexagon, polygon, and the like. Accordingly, the exterior surface of the attachment member may include an external surface configuration that is adapted for being inserted within or correspond to a receiving portion of an instrument so as to be received within the instrument and thereby function to attach the guide sleeve to the instrument.

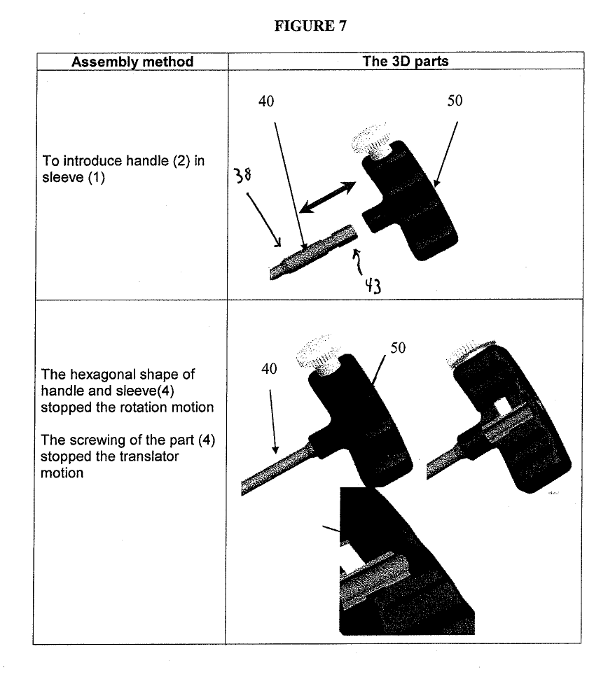

For example, as depicted in FIG. 7, the proximal portion (40) has an external configuration that is of a dimension for being at least partially received within an instrument that has a receiving portion with a complementary, shape thereto. The proximal portion (38) has a hexagonal (43) external shape positioned on the proximal end (40) of the guide sleeve. Thus, an additional device to be associated with the proximal end (40) preferably includes a complementary shape to the external shape of the proximal end (e.g., in this instance, hexagonal), in order for the additional device or agent to be associated with the proximal end attachment member. For instance, where the attachment member and additional instrument to be associated therewith have complimentary male to female configurations, the end portions of the two devices may be inserted one into the other. As depicted, a groove (41) is positioned between the hexagonal external shape (43) and the cylindrical portion of the elongated tubular body (10). In some embodiments, the groove (41) functions so as to contact a stop screw of an additional instrument (e.g., the stop screw of the handle (50)) that may be received and associated with the proximal portion (40) of the guide sleeve for the purpose of retaining the received instrument. In this manner, translation between the guide sleeve and the additional instrument, e.g., handle, may be avoided.

Thus, where a portion of an additional instrument includes a female attachment portion, this portion may be configured to receive the external attachment member of the proximal portion of the guide sleeve within the female attachment portion. Specifically, as previously described above, a handle (50) may be attached to the proximal end (40) of the guide sleeves of the present disclosure, and as illustrated in FIG. 7, the handle (50) may be specifically configured to be complementary to the external configuration of the attachment member (45) of the proximal end (40). As such, the proximal end of the subject guide sleeve has a specific shape which is complementary to the shape of the handle for easy attachment. For example, the handle and sleeve may both have complementary hexagonal shapes. As such, the handle enables a user to rotate the guide sleeve which in turn results in the insertion and fixation of the guide sleeve into the bone.

Some embodiments of the subject disclosure are directed toward methods of use and methods of treatment for the guide sleeve are provided. In some embodiments, methods of employing the subject guide sleeve in the performance of various therapeutic treatments are provided herein. Specifically, in some embodiments, the guide sleeve may be used to more precisely inject, place, apply, dispense or otherwise administer additional devices, agents, and instruments into the vertebral body.

For example, the subject guide sleeve may be used in vertebroplasty or kyphoplasty procedures, such as in an image-guided, minimally invasive, nonsurgical procedure wherein the guide sleeve is used in conjunction with other instruments to strengthen a broken vertebra (spinal bone) that has been weakened so as to prevent vertebral collapse. Generally, in some embodiments, the guide sleeve may be used in a vertebroplasty procedure to provide a percutaneous access to a vertebral body and/or a cavity thereof, whereby an instrument, such as an injection instrument containing an injection applicator, may be affixed to and/or guided through the guide sleeve so as to inject an orthopedic cement mixture through the applicator into the fractured bone and/or a cavity thereof. In other embodiments, the subject guide sleeve may be employed in a kyphoplasty procedure.

In a typical vertebroplasty procedure, a patient is initially placed in a prone position so that the affected vertebral body is within a field of an imaging device, such as an X-ray projection fluoroscopy imaging device. The skin overlying the vertebral body is prepped and draped in the usual manner utilizing acceptable sterile technique. A suitable anesthetic is injected into the periosteum of a selected pedicle to be entered, as well as into the surrounding area. Using a scalpel, a skin incision of about five millimeters in length is made, and the selected pedicle is initially exposed.

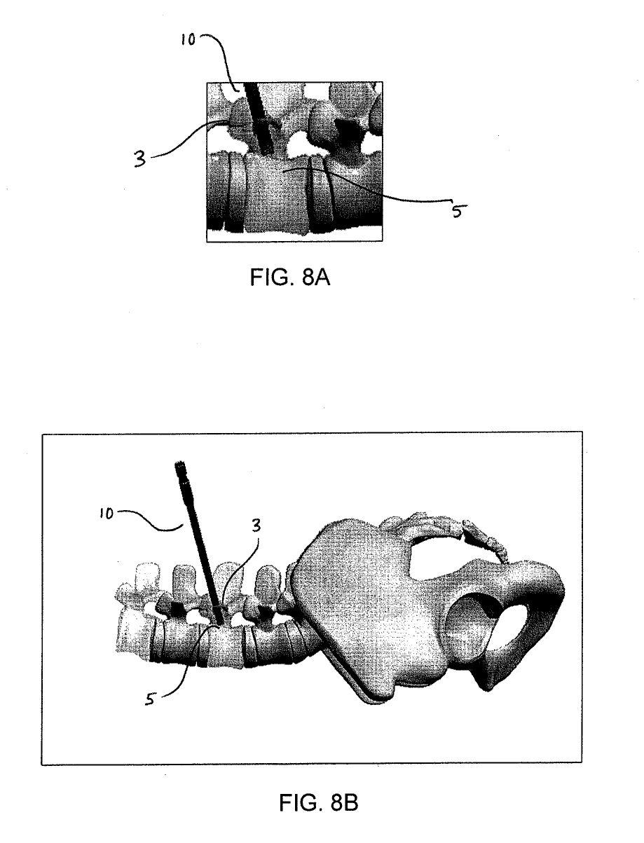

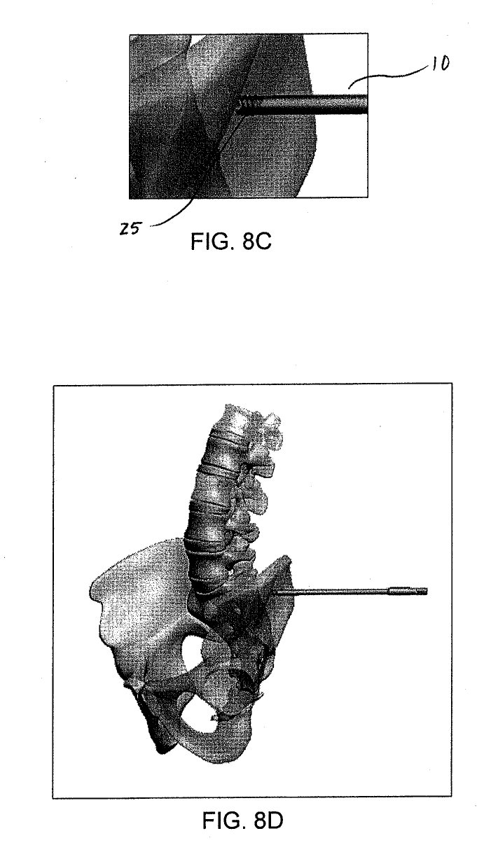

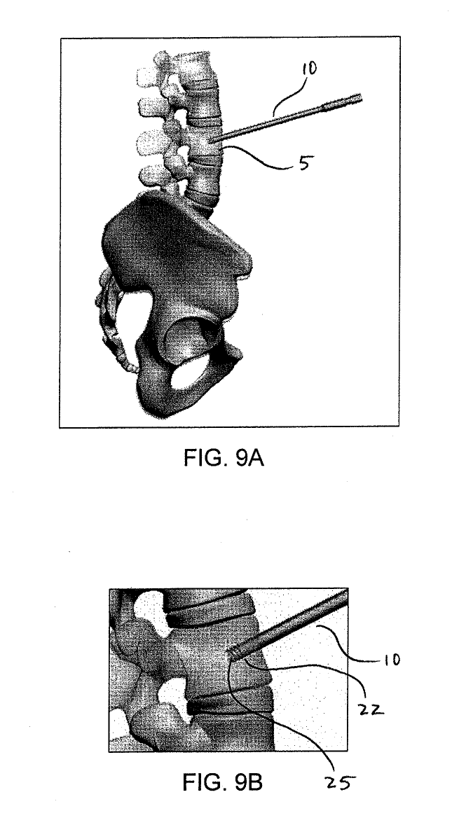

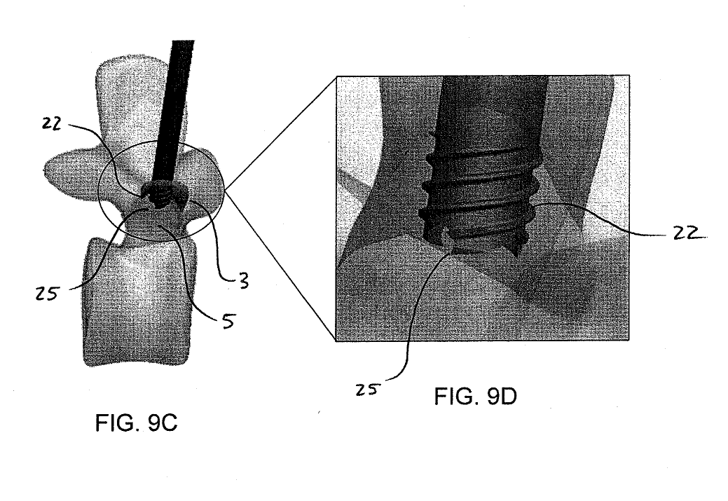

As illustrated in FIGS. 8A and 8B, a guide sleeve (10) of the subject disclosure, is inserted into the incision (not shown), and passed down a selected pedicle (3) until it enters the vertebral body (5), see for instance, FIG. 8A. As depicted, the guide sleeve (10) is inserted at the entry of the pedicle. The insertion is approximately 5 mm in depth. A handle (not shown) operatively attached to the guide sleeve (10) is then rotated until the penetration member (25) penetrates the vertebral bone (5) pedicle and the fixation element (22) attaches thereto and thereby locks the guide sleeve (10) to the bone (5), see for instance, FIG. 8B. Specifically, in some embodiments, the rotational motion causes the penetration member (25) and fixation elements (22) of the subject guide sleeve (10) to penetrate, affix and attach to the specific treatment site, e.g., in the vertebral bone (5) to be treated. See, for instance, FIGS. 9C and 9D.

The rotation or screwing of the guide sleeve may be stopped when the sleeve is firmly attached to the pedicle. For instance, as depicted in FIGS. 8C and 8D, the guide sleeve is inserted until it is in contact with the selected portion, such as an iliac crest or other bone portion. Then, it is rotated into the bone, for instance, about 5 mm depth, until the guide sleeve is strongly, e.g., firmly, attached to the bone.

Alternatively, as illustrated in FIGS. 9A through 9D, a vertebral body may be accessed by transformational approach or anterior approach or antero-lateral approach. The guide sleeve may be inserted until it is in contact with the vertebral body wall. Then, it is rotated into the bone, e.g., about 3 mm depth, depending on the vertebral wall thickness, until the guide sleeve is firmly attached to the bone.

By rotating the subject guide sleeve, the teeth positioned at the distal portion penetrate into the bone and the threaded portion positioned coextensively with the teeth at the distal portion of the guide sleeve enter the opening formed by the teeth thereby becoming affixed thereto and consequently attaching the guide sleeve to the bone. The lumen of the tubular member of the subject guide sleeve is specifically configured so that a vertebroplasty device (for example) may be guided through the guide sleeve and into the access created thereby so as to access the site of treatment. As such, the vertebroplasty device may then be inserted into the guide sleeve and guided within the lumen to the specific treatment site. A cement injector may then be attached to the proximal end of the subject guide sleeve and the cement is thereafter injected into the vertebral body. Once it is determined, e.g., through X-ray visualization, that a sufficient amount of cement has been injected to provide the desired strength and height so as to restore the vertebral body, the treatment is completed, the instruments and guide sleeve are removed, and the incision closed. Many of the above-noted steps may also be applied in a kyphoplasty procedure.

In some embodiments, the subject guide sleeve may be used in the delivery of intersomatic cages and/or screws, for a spinal fusion procedure. For example, an entry site may be created in the patient's back along the portion of the spine to be treated. The spine and disk space are then exposed for treatment. The intervertebral body space may be accessed by posterior approach or transformational approach or anterior approach or antero-lateral approach. The guide sleeve is inserted until it is in contact with the vertebral body bone from the upper and lower vertebrae. Then, it is rotated into the bone/endplates, for instance, at about 3 to 5 mm depth, until the guide sleeve is firmly attached to the bone. The disk space between vertebrae to be fused is then cleaned, leaving as much as possible of the disk annulus in place to facilitate retaining the bone graft material and any fluids delivered to the area within the original disk space. Bone graft or a bone graft substitute may then be inserted, or packed, into a portion of disk space, leaving sufficient room for the insertion of one or more interbody cages.

In some embodiments, the lumen of the tubular member of the subject guide sleeve is specifically configured so that the interbody cage or screw may be guided there through to site of treatment. As such, the interbody cage or screw is then inserted into the guide sleeve and guided within the lumen to the specific treatment site. The cage and screw may be inserted from the posterior of the patient to one side of the vertebral midline, and positioned within disk space adjacent the previously inserted bone graft material. Once inserted, the cage may be positioned approximately between the adjoining vertebrae.

Still other embodiments of the present disclosure provide kits for providing the subject devices and for use in practicing spinal treatment methods. Such kits may include at least one of the subject guide sleeves, as well as other implants for a specific type of spinal procedure. For example, certain kits may include a vertebroplasty device, a kyphoplasty device, intersomatic cages, screws, and the like. Additionally, the kits may include other instruments which are intended to be attached to the subject guide sleeves. For example, the kits may include an awl, drill, template, implant holder or an injection tube. Additionally, the kits may further include instructions for using the subject guide sleeves in therapeutic treatments.

Although the foregoing disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

What is claimed is:

1. A guide sleeve, comprising:
an elongate tubular body comprising a distal portion with a distal end, a proximal portion with a proximal end, and a lumen between said distal and proximal ends,
a penetration member, positioned on said distal portion and configured for penetrating a bone portion,
a fixation element, positioned at said distal portion and configured for fixing said sleeve to said bone portion.

2. The guide sleeve of claim 1, wherein said penetration member is coextensive with said tubular elongate member.

3. The guide sleeve of claim 1, wherein said penetration member comprises one or more teeth elements.

4. The guide sleeve of claim 3, wherein said one or more teeth elements are configured as a cut-out portion of said distal end of the elongate tubular body.

5. The guide sleeve of claim 4, wherein said cut out portion comprises a serrated edge.

6. The guide sleeve of claim 1, wherein said fixation element comprises threading.

7. The guide sleeve of claim 6, wherein said threading is coextensive with said elongate tubular body and said penetration member.

8. The guide sleeve of claim 1, wherein said proximal portion comprises an attachment member.

9. The guide sleeve of claim 8, wherein said attachment member is adapted for receiving an instrument member and thereby attaching said instrument member to the guide sleeve.

10. The guide sleeve of claim 9, wherein said attachment member comprises a male insertion element that is adapted for receiving a female receptacle element of said instrument member.

11. The guide sleeve of claim 10, wherein said attachment member is configured as a cleft region.

12. The guide sleeve of claim 8, wherein said instrument member comprises a handle.

13. The guide sleeve of claim 1, wherein said proximal end comprises a hexagonal configuration.

14. The guide sleeve of claim 13, wherein said hexagonal configuration comprises a hex-nut configuration.

15. The guide sleeve of claim 1, wherein a proximal portion of said lumen is configured for attaching to an additional instrument.

16. The guide sleeve of claim 15, wherein said additional instrument is at least an awl, drill, template, implant holder or cement injection tube.

17. The guide sleeve of claim 1, wherein said internal lumen is configured for guiding a device or agent through the elongate tubular member.

18. The guide sleeve of claim 17, wherein said device is selected from a vertebroplasty device, a kyphoplasty device, intersomatic cages, or screws.

19. The guide sleeve of claim 18, wherein the inner shape of the lumen is circular.

20. A method of affixing a guide sleeve to a vertebral bone, said method comprising the steps of:
providing a guide sleeve, wherein the guide sleeve comprises:
an elongate tubular body comprising a distal portion with a distal end, a proximal portion with a proximal end, and a lumen between said distal and proximal ends,
a penetration member, positioned on said distal portion and configured for penetrating a bone portion,
a fixation element, positioned at said distal portion and configured for fixing said sleeve to said bone portion;
making an incision in a tissue of a subject so as to expose a treatment site, wherein said treatment site comprises a portion of said vertebral bone;
inserting said guide sleeve through said incision so that said distal portion of the guide sleeve contacts said vertebral bone;
manipulating the guide sleeve in a manner sufficient to affix the guide sleeve to the vertebral bone.

21. A method of treating a patient suffering from a compression fracture within a vertebral bone, said method comprising the steps of:
providing a guide sleeve, wherein the guide sleeve comprises:
an elongate tubular body comprising a distal portion with a distal end, a proximal portion with a proximal end, and a lumen between said distal and proximal ends,
a penetration member, positioned on said distal portion and configured for penetrating a bone portion,
a fixation element, positioned at said distal portion and configured for fixing said sleeve to said bone portion;
making an incision in a tissue of a subject so as to expose a treatment site, wherein said treatment site comprises a portion of said vertebral bone;
inserting said guide sleeve through said incision so that said distal portion of the guide sleeve contacts said vertebral bone;
manipulating the guide sleeve in a manner sufficient to create an access in the vertebral bone and to affix the guide sleeve to the vertebral bone;
inserting an instrument through said lumen of the guide sleeve in a manner sufficient to cause at least a portion of said instrument to contact said access;
employing the instrument in a manner sufficient to perform a restoration procedure thereby treating the patient.

22. The method according to claim 21, wherein said instrument comprises a vertebroplasty instrument and said restoration procedure comprises vertebroplasty.

23. The method according to claim 21, wherein said instrument comprises a kyphoplasty instrument and said restoration procedure comprises kyphoplasty.

24. The method according to claim 21, further comprising using the guide sleeve to deliver one or more of a intersomatic cage or screw to said treatment site.

25. A kit for use in treating a patient suffering from a vertebral compression fracture, said kit comprising:
a guide sleeve according to claim 1; and
an additional treatment device.

26. A method of treating a patient suffering from a bone fracture within the iliac crest or other bone, said method comprising the steps of:
providing a guide sleeve, wherein the guide sleeve comprises:
an elongate tubular body comprising a distal portion with a distal end, a proximal portion with a proximal end, and a lumen between said distal and proximal ends,
a penetration member, positioned on said distal portion and configured for penetrating an iliac crest or other bone portion,
a fixation element, positioned at said distal portion and configured for fixing said sleeve to said iliac crest or other bone portion;
making an incision in a tissue of a subject so as to expose a treatment site, wherein said treatment site comprises a portion of said iliac crest or other bone;
inserting said guide sleeve through said incision so that said distal portion of the guide sleeve contacts said iliac crest or other bone;
manipulating the guide sleeve in a manner sufficient to create an access in the iliac crest or other bone and to affix the guide sleeve to the iliac crest or other bone.

27. The method according to claim 26, further comprising inserting an instrument through said lumen of the guide sleeve in a manner sufficient to cause at least a portion of said instrument to contact said access.

28. The method according to claim 27, further comprising employing the instrument in a manner sufficient to treat said bone fracture thereby treating the patient.

29. The method according to claim 26, further comprising using the guide sleeve to deliver one or more screw or plate or pin into said treatment site.

30. A kit for use in treating a patient suffering from a bone fracture, said kit comprising:
a guide sleeve according to claim 1; and
an additional treatment device.
